(19) United States
(12) Patent Application Publication
(10) Pub. No.: US 2014/0046111 A1
(43) Pub. Date: Feb. 13, 2014

(54) PROCESS FOR CONVERTING PARAFFIN WITH MODIFIED ZIRCONIA CATALYST

(71) Applicant: CPC Corporation, Taiwan, Taipei (TW)

(72) Inventors: Ying-Chieh Yang, Chiayi City (TW); Hung-Shan Weng, Chiayi City (TW); Shiann-Horng Chen, Chiayi City (TW); Cheng-Tsung Hong, Chiayi City (TW); Hung-Chung Shen, Chiayi City (TW)

(73) Assignee: CPC Corporation, Taiwan, Taipei (TW)

(21) Appl. No.: 14/056,176

(22) Filed: Oct. 17, 2013

Related U.S. Application Data
(62) Division of application No. 13/241,605, filed on Sep. 23, 2011.

(51) Int. Cl. C07C 5/27

(52) U.S. Cl. CPC C07C 5/2735
USPC 585/750

(57) ABSTRACT

A process for converting paraffin by using a modified zirconia catalyst is provided. The process has steps of: (i) providing feed containing n-pentane, n-hexane and more than 2 vol % n-heptane based on the volume of the feed; and (ii) subjecting the feed to isomerization of n-heptane with a modified zirconia catalyst comprising zirconium oxide, sulfate ions, a first metal component and a second metal component. Based on the weight of the catalyst, an amount of the first metal component ranges between 0.1 wt % and 15 wt %, an amount of the second metal component ranges between 0.2 wt % and 3.0 wt %, and an amount of the sulfate ions contain sulfur is less than 1.0 wt %. By using the modified zirconia catalyst with low sulfate content, the process is beneficial to improve iso-C₇ selectivity and conversion rate in heptane isomerization.

### CROSS REFERENCE TO RELATED APPLICATIONS

The application is a divisional application of U.S. patent application Ser. No. 13/241,605, filed on Sep. 23, 2011, and entitled “MODIFIED ZIRCONIA CATALYSTS AND ASSOCIATED METHODS THEREOF”. The content of the prior application is incorporated herein by its entirety.

### BACKGROUND OF THE INVENTION

1. Field of the Invention

The invention is related to a paraffin isomerization catalyst, particularly to a sulfated zirconia catalyst with aluminum, platinum and low sulfate content. The catalyst has high catalytic activity and is useful for producing isoparaffin-rich product in the paraffin isomerization process using heptane or paraffin that comprises heptane as feedstock.

2. Description of the Prior Arts

It is often taken into consideration whether the octane number of gasoline fits the safety requirements upon refining fuels to produce gasoline. Generally, the octane number represents the resistance of gasoline against auto-ignition (also called “knocking”). The higher octane number of gasoline represents better knocking resistance. Heptane, one of the components of petroleum, has a high tendency to burn explosively, so it was given an octane number “0”. On the other hand, isooctane does not easily burn explosively, such that it was given an octane number “100”. The branched-chain alkane usually has a higher octane number than that of its normal alkane. Such catalysts can be used to precede alkane reformation, isomerization or other reactions to produce gasoline having high octane rating.

During paraffin isomerization processes, the major feed includes n-pentane (n-C₅) and n-hexane (n-C₆) with a small amount of n-heptane (n-C₇). The catalysts are used to change normal alkanes to isomeric alkanes containing single side chain or multiple side chains through catalytic reactions in order to increase the octane number of gasoline. After the earliest paraffin isomerization system made by Neuitzescu and Dragan in 1933, which performed the isomerization of n-hexane and n-heptane using aluminum chloride catalysts at low temperature, the Friedel-Craft catalysts for paraffin isomerization was introduced. These kinds of catalysts are such liquid-state, homogeneous and acidic catalysts with high activity under low temperature; however, they are difficult to be separated from products and often make corrosion on equipment. A bifunctional catalyst, alumina-supported noble metal, which was developed in 1950, is a solid-state acidic catalyst containing transition metal. Due to the fact that the acidity of alumina is not strong enough, it is necessary for alumina catalysts to be added by silica or boron oxide in order to increase acidity and abate reaction temperature. It was found in the 1960s by Rabo et al. that large-porous acidic zeolite catalysts that have noble metal are thermally stable, having long lifetime along with good resistance against sulfur and nitrogen so that these zeolite catalysts [such as Y zeolite containing palladium (i.e. Pd/Y) or platinum (i.e. Pt/Y)] can make good use of isomerization processes of n-paraffin compounds. Mordenite catalysts containing platinum developed in 1970s have higher activity and selectivity of isoparaffin products than Pd/Y and Pt/Y and are widely used in manufacturing procedures.

A comparison of the following types of paraffin isomerization catalysts: chlorinated alumina, common zeolite, modern zeolite, and metal oxide is shown in Table 1, and these four catalysts described above all contain platinum. Alumina catalysts containing platinum namely Pt/Al₂O₃ (not shown in Table 1) solved the problems about difficult separation and apparatus corrosion, but they need considerably high operating temperature. The chlorinated alumina catalysts increase acidity of alumina thereof by using methane tetrachloride resulting in operable temperature between 130° C. and 150° C., and therefore overcome the drawback of high reaction temperature of the alumina catalysts; however, when using the chlorinated alumina catalysts in reaction, the feedstock must be supplied with chlorine frequently and kept away from water, sulfur and other oxygenates, or else it causes chloride corrosion on equipment. Furthermore, the lifetime of the chlorinated alumina catalysts is about 2 to 3 years and they are thereafter irreversibly depleted. The common zeolite catalysts have better resistance against sulfur and water but much weaker acidity than the chlorinated alumina catalysts. These properties of the common zeolite catalysts make high reaction temperature thereof up to between 260° C. and 280° C. According to the thermodynamic equilibrium distribution of n-C₆ and n-C₇, performing paraffin isomerization at low temperature can obtain more branched-chain alkanes such that the manufactured gasoline has higher octane rating. The modern zeolite catalysts still possess high reaction temperature between 250° C. and 280° C., though they have good sulfur and water resistance. The reaction temperature of the metal oxide (regarded as zirconia herein) catalysts is reduced by 60 to 70° C. as a result of their stronger acidity than that of the modern zeolite catalysts (Hua et al., Journal of Catalysis, 197, 406-413, 2001). Also, the resistance to sulfur and water of the metal oxide catalysts is higher than that of the chlorinated alumina catalysts. Thus, to improve properties and performance of the metal oxide catalysts is increasingly important in refinery economics.

Table 1 shows comparison among the four types of paraffin isomerization catalysts (chlorinated alumina, common zeolite, modern zeolite, and metal oxide) (Wevda and Kohler, Catalysis Today, 81, 51-55, 2003)

| | chlorinated | metal oxide | common | modern |
|---|---|---|---|---|
| Catalyst-process | alumina | (zirconia) | zeolite | zeolite |
| Feedstock | | | | |
| conditions | | | | |
| Feedstock type | C5/C6 | C5/C6 | C5/C6 | C5/C6 |
| Sulphur (ppm) | None | <20 | <20 | <200 |
| Water (ppm) | None | <20 | <20 | <200 |
| Aromatics/ | <2 | <2 | <2 | <10 |
| benzene (%) | | | | |
| C7⁺ (%) | <2 | <2 | <2 | <5 |
| Feed-product | | | | |
| treament | | | | |
| HDS | Yes | Yes | Yes | Optional |
| Sulphur Guard | Yes | Optional | Optional | None |
| Feed dryer | Yes | Optional | Optional | None |
| Hydrogen dryer | Yes | Optional | Optional | None |
| Effluent guard | Yes | None | None | None |
| system | | | | |
| Typical process | | | | |
| condition | | | | |
| Temperature (° C.) | 130-150 | 180-210 | 260-280 | 250-280 |
| Pressure (barg) | 15-35 | 15-35 | 15-35 | 15-35 |
| LHSV (h⁻¹) | 1-3 | 1-3 | 1-3 | 1-3 |
| H₂/HC-molar ratio | 186 | 178-186 | 168-186 | 172-186 |
| Typical isomerate | | | | |
| properties | | | | |
| i-C5/C5ratio^{a} | 68-72 | 65-71 | 60-65 | 63-67 |
| 2,2-DMB/C6ratio^{a} | 21 | 20.5 | 16 | 19 |
| Isomerate yield^{b} (%) | 96+ | 96+ | 95+ | 96+ |
| Isomerate octane | Up to 94 | Up to 94 | Up to 94 | Up to 94 |

^{a}Reactor outlet
^{b}Unit outlet

The feed of paraffin isomerization processes usually contains n-C₅, n-C₆ and a small amount of n-C₇. Various types of catalysts cannot effectively convert C₅-C₇ alkanes at the same time. Catalysts mostly have a high conversion rate of C₅/C₆ isomerization reactions; however, these catalysts also make considerably high cracking effect on C₇ alkanes during isomerization resulting in carbon accumulation therein and catalyst degradation. Thus, it is necessary to limit the C₇ content of the feed. As shown in Table 1, the C₇ content is allowed to be only up to 5 vol %.

Using Pt-promoted sulfated zirconia catalysts (Pt/SZ catalysts) which have stronger acidity and need lower reaction temperature than zeolite catalysts for paraffin isomerization processes is beneficial to increase yield of isoparaffin with multiple side chains. Therefore, the gasoline products produced through Pt/SZ catalysts often have higher octane number. Pt/SZ catalysts also have higher resistance to water and sulfur than chlorinated alumina catalyst containing platinum (Pt/AlCl₃ catalyst). For C₅/C₆ isomerization reaction, Pt/SZ catalysts possess superior properties compared with other kinds of catalysts; however, Pt/SZ catalysts can bring severe cracking effects on C₇ alkanes upon C₇ isomerization reaction.

Iglesia et al. performed such experiments on applying Pt/SZ catalysts in paraffin isomerization and found that the cracking/isomerization ratio of the products while using C₇ alkanes as the feed was 40 times higher than using C₅/C₆ alkanes as the feed (Journal of Catalysis, 144, 238-253, 1993). As shown in Table. 2, Miyaji et al. made comparison of catalytic activity and isoparaffin Pd—WO₃/ZrO₂ catalysts, Pt—SO₄²⁻/ZrO₂ catalysts and Pt/H-β zeolite catalysts, wherein Pt—SO₄²⁻/ZrO₂ catalysts have lowest i-C₇ selectivity (Applied Catalysis, 262, 143-148, 2004). Grau et al. impregnated AlCl₃ with platinum, then mixed physically with sulfate zirconia catalysts in order to prevent platinum and the acidic groups of the sulfate zirconia catalysts from physical and chemical reactions, but the tendency and selectivity toward paraffin cracking are still much higher than that toward paraffin isomerization (Applied Catalysis, 172, 311-326, 1998). Bouchenafa-Saïb et al. used montmorillonite to modify the sulfated zirconia catalysts causing the reduction of the products made by C₇ cracking, but the activity of the sulfated zirconia catalysts modified by montmorillonite also decreased and thus the reaction temperature increased by more than 80° C. The sulfated zirconia catalysts modified by montmorillonite needed a reaction temperature of up to 350° C. in order to achieve an overall conversion rate of 70% (Applied Catalysis, 259, 9-15, 2004).

In view of the prior art described above, some catalysts only have high catalytic activity for paraffin isomerization at high temperature, while other catalysts with high activity have high tendency to cause the cracking of C₇ alkanes. Thus, these catalysts according to prior art may cause waste of energy and materials, catalyst degradation due to carbon accumulation as well as decrease of manufacturing efficiency. The sulfated zirconia catalysts mentioned above all contain a certain level of sulfur content resulting in strong acidity thereof. For n-heptane isomerization, such catalysts according to prior art still cannot have high activity and selectivity of isoparaffin products at low temperature and cannot avoid undesired cracking. Therefore, Catalysts with both high i-C₇ selectivity and catalytic activity are urgently demanded at present.

Table 2 shows comparison among the four types of paraffin isomerization catalysts below: Pd-10 wt % HSiW/SiO₂, Pd-40 wt % HSiW/SiO₂, Pd—H-β zeolite, Pt—SO₄²⁻/ZrO₂, Pt/H-β zeolite and Pd—WO₃/ZrO₂ (Applied Catalysis, 262, 143-148, 2004)

| | | Pd-10 wt % | Pd-40 wt % | Pd—H-β | Pt—SO₄^{2-/} | Pt/H-β | Pd—WO₃/ |
|---|---|---|---|---|---|---|---|
| Catalyst^{a} | | HSiW/SiO₂ | HSiW/SiO₂ | zeolite | ZrO₂ | zeolite | ZrO₂ |
| Conversion (%) | | 69.2^{b} | 59.7^{b} | 66.0^{c} | 71.8^{b} | 57.3^{b} | 67.9^{c} |
| Selectivity^{d} (mol %) | | | | | | | |
| Cracking | C₁ + C₂ | 0 | 0 | 0 | 0 | 0 | 0 |
| products | | | | | | | |
| | C₃ + iso-C₄ | 5.4 | 37.3 | 3.4 | 6.0 | 74.1 | 4.1 |
| | C₅ + C₆ | 0 | 0 | 0 | 0 | 0 | 0 |
| Monobranched | 2-MH (42) | 35.4 | 23.5 | 37.7 | 35.4 | 10.2 | 35.7 |
| C₇^{e} | | | | | | | |
| | 3-MH (52) | 35.5 | 22.0 | 36.0 | 32.9 | 10.1 | 33.0 |
| | 3-EP (65) | 2.2 | 0 | 2.3 | 1.8 | 0 | 2.0 |
| Multibranched | 2,2-DMP | 5.5 | 2.3 | 5.5 | 7.1 | 0 | 6.8 |
| C₇^{f} | (98) | | | | | | |
| | 2,3-DMP | 8.3 | 7.5 | 6.8 | 7.1 | 2.9 | 8.6 |
| | (91) | | | | | | |
| | 2,4-DMP | 8.1 | 7.4 | 6.1 | 7.2 | 2.7 | 8.5 |
| | (83) | | | | | | |
| | 3,3-DMP | 1.2 | 0 | 2.2 | 2.5 | 0 | 1.3 |
| | (81) | | | | | | |
| | 2,2,3-TMB | 0.4 | 0 | 0 | 0 | 0 | 0 |
| | (112) | | | | | | |
| Total branched C₇ | | 94.6 | 62.7 | 96.6 | 94.0 | 25.9 | 95.9 |

Reaction temperature: 453 K, C₇:H₂ = 4.8:95.2.
^{a}The loading amount of Pd or Pt was 2 wt %.
^{b}Total flow rate(F): 20 ml (W/F = 20 gh mol⁻¹).
^{c}Total flow rate (F): 10 ml (W/F = 40 gh mol⁻¹).
^{d}100 × n[Cₙ]/[total carbon atom], where [Cₙ] and [total carbon atom]indicate concentration of hydrocarbon having n carbon atoms and total carbons, respectively.
^{e}2-MH, 3-MH and 3-EP refer to 2-and 3-methylhaxane, and 3-ethylpentane, respectively.
^{f}2,2-DMP, 2,3-DMP, 2,4-DMP, 3,3-DMP and 2,2,3-TMB refer to 2,2-, 2,3-, 2,4-, and 3,3-dimethylpentanes, and 2,2,3-trimethylbutane, respectively.
The number in parenthesis is the research octane number.

U.S. Pat. No. 7,041,866 discloses a sulfated zirconia catalyst containing at least one of the platinum-group metal elements and optionally containing gallium, indium or ytterbium, etc. that provides the advantages of high activity, improved stability and increasing yields of converting light naphtha to desired and higher-octane isoparaffin products. However, the sulfur content of the catalyst is between 0.5 wt % and 5 wt % and the sulfur source is unknown.

U.S. Pat. No. 7,015,175 discloses a sulfated zirconia catalyst containing at least one of the platinum-group metal elements, and at least one of the lanthanide elements or ytterbium, yttrium, and optionally adding inorganic-oxide binder. The catalyst provides extra increased ring-opening activity, yet such catalyst must contains 0.01-10 wt % of the at least one of lanthanide elements or yttrium, etc.,

U.S. Pat. No. 6,448,198 discloses a method for manufacturing a sulfated zirconia catalyst. The catalyst produced by the method taught in the cited patent has a surface area more than 150 m²/g, a pore area not less than 0.2 cm³/g and an average pore diameter not less than 2 nm. The catalyst has higher activity while using n-hexanes as the feed of isomerization processes. However, the sulfur content of the catalyst described above is between 1 wt % and 10 wt % based on the weight of zirconium.

U.S. Pat. No. 6,037,303 discloses a method for manufacturing a sulfated zirconia catalyst having distinctive pore properties and superior acidity, which is made by only one step. The produced catalyst has a tetragonal phase structure and only a single-layered sulfate thereon. Also, the catalyst has at least 70% of its pores possessing apertures ranging from 1 nm to 4 nm. The catalyst has 1-3 wt % of sulfur and 0.1-3.0 wt % of platinum. However, the catalyst disclosed in this patent contains more sulfur content than the modified zirconia catalyst of the present invention without any Group IIIA (IUPAC 13) metal elements (such as aluminum and gallium).

To overcome the shortcomings, the present invention provides a modified zirconia catalyst and associated method to mitigate or obviate the aforementioned problems.

### SUMMARY OF THE INVENTION

The purpose of the present invention is to provide a modified zirconia catalyst that contains aluminum and platinum with a low content of sulfate ions in order to improve the selectivity of isoheptane (i.e. i-C₇ selectivity) during heptane isomerization.

Accordingly, the present invention provides a modified zirconia catalyst comprising zirconium oxide, sulfate ions, a first metal component and a second metal component, wherein the first metal component contains at least one of Group A (IUPAC 13) metal elements or a combination thereof at an amount of between 0.1 wt % and 15 wt % based on the weight of the catalyst, the second metal component contains a substance selected from the group consisting of platinum, platinum oxide, palladium, palladium oxide and a combination thereof at an amount of between 0.2 wt % and 3.0 wt % based on the weight of the catalyst, and the sulfate ions contain sulfur at an amount of less than 1.0 wt % based on the weight of the catalyst.

In another aspect, the present invention provides a method for manufacturing a modified zirconia catalyst comprising steps of:
(i) providing a zirconium oxide precursor and a first metal precursor;
(ii) blending and mixing the zirconium oxide precursor and the first metal precursor to form a solution, and adjusting pH value of the solution to range from 6 to 8;
(iii) allowing the solution to stand to form precipitates, filtering the precipitates and removing impurities from the precipitates, then drying the precipitates;
(iv) providing a sulfate ion solution;
(v) impregnating the dried precipitates within the sulfate ion solution to obtain sulfated precipitates, wherein the sulfated precipitates has a content of sulfate ion between 1 wt % and 15 wt % based on the weight of the dried precipitates, then calcining the sulfated precipitates to obtain a first-calcined precipitates;
(vi) providing a second metal precursor solution;
(vii) impregnating the first-calcined precipitates with the second metal precursor solution, then calcining the impregnated first-calcined precipitates to obtain a modified zirconia catalyst.

In yet another aspect, the present invention provides a process for converting paraffin comprising the steps of:
(i) providing feed containing n-pentane, n-hexane and more than 2 vol % n-heptane based on the volume of the feed;
(ii) subjecting the feed to isomerization of n-heptane with the modified zirconia catalyst according to the present invention, wherein the i-C₇ selectivity is higher than 80% as the conversion rate rises to 80%.

It is known that the acidic strength of the sulfated zirconia catalysts containing platinum is higher than that of general zeolite catalysts and Pt/WOx-ZrO₂ catalysts. Strong acidity allows paraffin isomerization reaction to perform at lower temperature. As shown in Table 1, the sulfated zirconia catalysts need to perform reaction at a lower temperature than the zeolite catalysts. According to the thermodynamic equilibrium diagram of n-hexane, n-heptane and the isomers thereof, low temperature can lead to more formation of isomers with side chain and thus increase the octane number of gasoline products and economize energy. However, the sulfated zirconia catalysts make severe cracking effects on n-heptanes (as shown in Table 2) resulting in the limited applications.

It is desired to decrease the acidic strength and acid content of the catalysts to reduce cracking. In terms of sulfated zirconia catalysts, Föttinger and Katada et al. found that the sulfated zirconia catalysts with low sulfate content have poor activity, or are even inactive (Applied Catalysis, 284, 69-75, 2005; Journal of Physical Chemistry, B 104, 10321-10328, 2000). Also, Laizet et al. reported that catalysts used in n-hexanes isomerization must contain proper density of sulfate to perform high activity and selectivity of isoparaffin (Topics in Catalysis, 10, 89-97, 2000). Those references described above show that the sulfated zirconia catalysts containing platinum must have appropriate concentration of sulfate, otherwise sulfate content that is too low results in low, even none, activity of the catalysts. Nevertheless, one of the features of the invention is decreasing acidic strength of the catalyst by means of changing the source of sulfate ions and lowering sulfate content without diminishing activity. The modified zirconia catalyst in accordance with the invention can maintain high activity and increase selectivity of isoparaffin products. In addition, the present invention uses ammonium sulfate instead of sulfuric acid as the source of sulfate ions. Therefore, a sulfated zirconia catalyst decreasing the sulfate ions content and the acidic strength is provided in this invention. Another pioneering endeavor is adding appropriate amount of aluminum into the catalyst to modify the properties and improve the activity thereof.

Compared with the catalyst disclosed in the cited patents, the modified zirconia catalyst of the present invention lays specific emphasis on the sulfur source that the sulfur content of the modified zirconia catalyst of the present invention is less than 1.0 wt % and not need to contain any of lanthanide elements or yttrium disclosed by the cited patents. Furthermore, the modified zirconia catalyst of the present invention contains at least one of the Group A (IUPAC 13) metal elements (such as aluminum) to facilitate and maintain its activity. With regard to the modified zirconia catalyst of the present invention, the advantages include not only low sulfur content but also appropriate amount of aluminum (or gallium), which is beneficial to promote the catalytic activity thereof.

The modified zirconia catalyst of the present invention has the advantage of greatly improved i-C₇ selectivity. When the modified zirconia catalyst of the present invention reaches an overall conversion rate of 70% in the isomerization reaction, the i-C₇ selectivity can rise from 25% to 83% or more without decreasing the catalytic activity. Compared with the current commercial catalysts for paraffin isomerization, the modified zirconia catalyst of the present invention is provided with higher activity and better i-C₇ selectivity, for example, under an overall conversion rate of 80% in the isomerization, the modified zirconia catalyst of the present invention can perform reaction at a reaction temperature 50° C. lower, which result in higher i-C₇ selectivity than the commercial catalysts.

Accordingly, the present invention provides the modified zirconia catalyst having lower content of sulfate ions and uses ammonium sulfate as the source of the sulfate ions in order to decrease the acidic strength, improve the selectivity of i-C₇ and thus lower the cracking level of n-heptanes during isomerization reaction as well as maintain high activity. Furthermore, adding proper amount of aluminum into the modified zirconia catalyst of the present invention can maintain stable activity thereof.

Other objectives, advantages and novel features of the invention will become more apparent from the following detailed description when taken in conjunction with the accompanying drawings.

### BRIEF DESCRIPTION OF THE DRAWINGS

FIG. 1 is a plot showing the conversion rate of n-heptane isomerization of the catalysts made in the comparative examples 1 to 3 and examples 1 to 3 respectively versus the reaction temperature.

FIG. 2 is a plot showing the i-C₇ selectivity of the catalysts made in the comparative examples 1 to 3 and examples 1 to 3 respectively versus the conversion rate of n-heptane isomerization.

FIG. 3 is a plot showing the conversion rate of n-heptane isomerization of the catalyst made in the examples 4 to 7 respectively versus the reaction temperature.

FIG. 4 is a plot showing the i-C₇ selectivity of the catalysts made in the comparative examples 4 to 7 and examples 1 to 3 versus the conversion rate of n-heptane isomerization.

FIG. 5 is a plot showing the activity of the commercial catalyst and the 1.5Pt/3.0SZA catalyst respectively as the feed contains n-hexane and n-heptane (the volume ratio of C₆/C₇: 70/30) during isomerization process.

FIG. 6 is a plot showing the i-C₇ product yield of the reaction with 1.5Pt/3.0SZA catalyst and the commercial catalyst respectively versus the conversion rate of n-hexane isomerization.

FIG. 7 is a plot showing the multibranched i-C₇ product yield of reaction with the 1.5Pt/3.0SZA catalyst and the commercial catalyst respectively versus the conversion rate of n-hexane isomerization.

FIG. 8 is a plot showing the i-C₆ product yield of the reaction with 1.5Pt/3.0SZA catalyst and the commercial catalyst respectively versus the conversion rate of n-hexane isomerization.

### DETAILED DESCRIPTION OF THE PREFERRED EMBODIMENTS

The present invention provides a modified zirconia catalyst comprising zirconium oxide, sulfate ions, a first metal component and a second metal component, wherein the first metal component contains at least one of Group A (IUPAC 13) metal elements or a combination thereof at an amount of between 0.1 wt % and 15 wt % based on the weight of the catalyst, the second metal component contains a substance selected from a group consisting of platinum, platinum oxide, palladium, palladium oxide and a combination thereof at an amount of between 0.2 wt % and 3.0 wt % based on the weight of the catalyst, and the sulfate ions contain sulfur at an amount of less than 1.0 wt % based on the weight of the catalyst.

In a preferred embodiment of the present invention, the amount of the first metal component is between 0.1 wt % and 10 wt % based on the weight of the catalyst.

In a preferred embodiment of the present invention, the source of the sulfate anions comprises ammonium sulfate, sulfuric acid, other compounds containing sulfate ions or a combination thereof, and more particularly is ammonium sulfate.

In a preferred embodiment of the present invention, the first metal component comprises a substance selected from the group consisting of aluminum, gallium and a combination thereof, and more particularly is aluminum.

In a preferred embodiment of the present invention, the second metal component is platinum.

In a preferred embodiment of the present invention, the zirconium oxide is ZrO₂.

In a preferred embodiment of the present invention, the BET specific surface area of the catalyst ranges from 50 m²/g to 130 m²/g.

In another aspect, the present invention provides a method for manufacturing a modified zirconia catalyst comprising steps of:
(i) providing a zirconium oxide precursor and a first metal precursor;
(ii) blending and mixing the zirconium oxide precursor and the first metal precursor to form a solution, and adjusting the pH value of the solution to range from 6 to 8;
(iii) allowing the solution to stand to form precipitates, filtering the precipitates and removing impurities from the precipitates (by washing or other available means), then drying the filtered precipitates;
(iv) providing a sulfate ion solution;
(v) impregnating the dried precipitates within the sulfate ion solution to obtain sulfated precipitates, wherein the sulfated precipitates has a content of sulfate ions between 1 wt % and 15 wt % based on the weight of the dried precipitates, then calcining the sulfated precipitates to obtain a first-calcined precipitates;
(vi) providing a second metal precursor solution;
(vii) impregnating the first-calcined precipitates within the second metal precursor solution, then calcining the impregnated first-calcined precipitates to obtain a modified zirconia catalyst.

In a preferred embodiment of the present invention, the first metal precursor contains a substance selected from a group consisting of aluminum compound, gallium compound, other compounds containing at least an element of Group A and a combination thereof, and more particularly is aluminum compound.

In a preferred embodiment of the present invention, the second metal precursor contains a substance selected from a group consisting of platinum compound, palladium compound and a combination thereof, and more particularly is platinum compound.

In a preferred embodiment of the present invention, the zirconium oxide precursor contains a substance selected from a group consisting of ZrOCl₂, ZrO(NO₃)₂, ZrOSO₄, ZrO(OH)NO₃ and a combination thereof, or other alternative compounds, and more particularly is ZrOCl₂.

In yet another aspect, the present invention provides a process for converting paraffin comprising the steps of:
(i) providing feed containing n-pentane, n-hexane and more than 2 vol % n-heptane based on the volume of the feed;
(ii) subjecting the feed to isomerization of n-heptane with the modified zirconia catalyst according to the present invention wherein the i-C₇ selectivity is higher than 80% as the conversion rate rises to 80% thereof.

The following examples serve to illustrate certain specific embodiments of the present invention. These examples should not, however, be construed as limiting the scope of the invention as set forth. There are many possible other variations that those of ordinary skill in the art will recognize, which are within the scope of the invention.

### Examples

### Preparation of the Modified Zirconia Catalyst

Ten different examples are provided below and sorted into three groups: (1) comparative examples 1 to 3 show preparation of sample catalysts according to prior art by impregnation with different contents of sulfate at presence of a certain content of platinum without adding aluminum for reference to and comparison with the invention; (2) examples 1 to 3 show preparation of sample catalysts according to the invention by impregnation with different contents of sulfate at presence of a certain content of platinum; (3) examples 4 to 7 show preparation of sample catalysts according to the invention by impregnation with different contents of sulfate at presence of a certain content of platinum. These sample catalysts produced in examples 1 to 7 all contain aluminum and can be regarded as a series of comparison of the modified zirconia catalyst of the present invention, wherein the sample catalyst made in example 5 is the best embodiment.

### Comparative Example 1

### The Preparation of 0.3Pt/1.5SZ Catalyst According to Prior Art

The sample catalyst is made by the following steps:
(i) dissolving 10 g zirconyl chloride octahydrate (ZrOCl₂8H₂O, marketed by J.T.Baker) in 100 ml distilled water, then mixing adequately to form a solution;
(ii) adding 25 wt % of ammonia [NH₃(aq), marketed by J.T.Baker] to adjust the pH value of the solution to 9.0.
(iii) allowing the solution to stand for 3 hours to form precipitates, filtering the precipitates and removing impurities (such as chloride ions) from the precipitates by washing with deionized water, then drying the filtered precipitates at 160° C. for 16 hours;
(iv) providing a ammonium sulfate solution [(NH4)₂SO₄(aq), marketed by J.T.Baker];
(v) impregnating the dried precipitates in the ammonium sulfate solution to obtain sulfated precipitates, wherein the sulfated precipitates has a sulfate ions content of 1.5 wt % based on the weight of the dried precipitates, then desiccating the sulfated precipitates at 100° C. and calcining the sulfated precipitates at 650° C. for the first time to obtain a sulfated zirconia catalyst, which is denoted as 1.5SZ catalyst;
(vi) impregnating the 1.5SZ catalyst obtained from step (v) into chloroplatinic acid hexahydrate (H₂PtCl₆6H₂O, marketed by Sigma-Aldrich);
(vii) drying the 1.5SZ catalyst that is impregnated into chloroplatinic acid hexahydrate at 100° C., then calcining it at 500° C. for 3 hours to obtain a sulfated zirconia catalyst containing 0.3 wt % of platinum based on the weight of the calcined 1.5SZ catalyst), which is denoted as 0.3Pt/1.5SZ.

### Comparative Example 2

### The Preparation of 0.3Pt/3.0SZ Catalyst According to Prior Art

The sample catalyst is made by the steps as described in comparative example 1, except that step (v) is impregnating the dried precipitates in the ammonium sulfate solution to obtain sulfated precipitates such that the sulfated precipitates has a sulfate ion content of 3.0 wt % based on the weight of the dried precipitates. The sample catalyst made in this example is denoted as 0.3Pt/3.0SZ.

### Comparative Example 3

### The Preparation of 0.3Pt/9.0SZ Catalyst According to Prior Art

The sample catalyst is made by the steps as described in comparative example 1, except that step (v) is impregnating the dried precipitates in the ammonium sulfate solution to obtain sulfated precipitates such that sulfated precipitates has a sulfate ion content of 9.0 wt % based on the weight of the dried precipitates. The sample catalyst made in this example is denoted as 0.3Pt/9.0SZ.

### Example 1

### The Preparation of 0.3Pt/1.5SZA Catalyst According to the Present Invention

The sample catalyst is made by the following steps:
(i) blending 10 g zirconyl chloride octahydrate (ZrOCl₂8H₂O, marketed by J.T.Baker) and a proper amount of aluminum nitrate 9-hydrate [Al(NO₃)9H₂O, marketed by J.T.Baker], then dissolving them in 100 ml distilled water and mixing adequately to form a solution, wherein the solution contains about 5 mol % of alumina;
(ii) adding 25 wt % of ammonia [NH₃(aq), marketed by J.T.Baker] to adjust the pH value of the solution to 9.0.
(iii) allowing the solution to stand for 3 hours to form precipitates, filtering the precipitates and removing impurities (such as chloride ions) from the precipitates by washing with deionized water, then drying the filtered precipitates at 160° C. for 16 hours;
(iv) providing a ammonium sulfate solution [(NH₄)₂SO₄(aq), marketed by J.T.Baker];
(v) impregnating the dried precipitates in the ammonium sulfate solution to obtain sulfated precipitates, wherein the sulfated precipitates has a sulfate ion content of 1.5 wt % based on the weight of the dried precipitates, then desiccating the sulfated precipitates at 100° C. and calcining the sulfated precipitates at 650° C. for the first time to obtain a sulfated zirconia catalyst, denoted as 1.5SZA catalyst;
(vi) impregnating the 1.5SZA catalyst obtained from step (v) into chloroplatinic acid hexahydrate (H₂PtCl₆6H₂O, marketed by Sigma-Aldrich);
(vii) drying the 1.5SZA catalyst impregnated into chloroplatinic acid hexahydrate at 100° C., then calcining it at 500° C. for 3 hours to obtain a sulfated zirconia catalyst containing 0.3 wt % of platinum based on the weight of the calcined 1.5SZ catalyst, denoted as 0.3Pt/1.5SZA.

### Example 2

### The Preparation of 0.3Pt/3.0SZA Catalyst According to the Invention

The sample catalyst is made by the steps as described in example 1, except that step (v) is impregnating the dried precipitates in the ammonium sulfate solution to obtain sulfated precipitates such that the sulfated precipitates has a sulfate ion content of 3.0 wt % based on the weight of the dried precipitates. The sample catalyst made in this example is denoted as 0.3Pt/3.0SZA.

### Example 3

### The Preparation of 0.3Pt/9.0SZA Catalyst According to the Invention

The sample catalyst is made by the steps as described in example 1, except that step (v) is impregnating the dried precipitates in the ammonium sulfate solution to obtain sulfated precipitates such that the sulfated precipitates has a sulfate ion content of 9.0 wt % based on the weight of the dried precipitates. The sample catalyst made in this example is denoted as 0.3Pt/9.0SZA.

### Example 4

### The Preparation of 1.0Pt/3.0SZA Catalyst According to the Invention

The sample catalyst is made by the steps as described in example 1, except that step (v) is impregnating the dried precipitates in the ammonium sulfate solution to obtain sulfated precipitates such that the sulfated precipitates has a sulfate ion content of 3.0 wt % based on the weight of the dried precipitates, and altering the amount of platinum impregnation such that the platinum content of the sample catalyst is 1.0 wt % based on the weight of the calcined 3.0SZA catalyst. The sample catalyst made in this example is denoted as 1.0Pt/3.0SZA.

### Example 5

### The Preparation of 1.5Pt/3.0SZA Catalyst According to the Invention

The sample catalyst is made by the steps as described in example 1, except that step (v) is impregnating the dried precipitates in the ammonium sulfate solution to obtain sulfated precipitates such that the sulfated precipitates has a sulfate ion content of 3.0 wt % based on the weight of the dried precipitates, and altering the amount of platinum impregnation such that the platinum content of the sample catalyst is 1.5 wt % based on the weight of the calcined 3.0SZA catalyst. The sample catalyst made in this example is denoted as 1.5Pt/3.0SZA.

### Example 6

### The Preparation of 2.0Pt/3.0SZA Catalyst According to the Invention

The sample catalyst is made by the steps as described in example 1, except that step (v) is impregnating the dried precipitates in the ammonium sulfate solution to obtain sulfated precipitates such that the sulfated precipitates has a sulfate ion content of 3.0 wt % based on the weight of the dried precipitates, and altering the amount of platinum impregnation such that the platinum content of the sample catalyst is 2.0 wt % based on the weight of the calcined 3.0SZA catalyst. The sample catalyst made in this example is denoted as 1.0Pt/3.0SZA.

### Example 7

### The Preparation of 2.5Pt/3.0SZA Catalyst According to the Invention

The sample catalyst is made by the steps as described in example 1, except that step (v) is impregnating the dried precipitates in the ammonium sulfate solution to obtain sulfated precipitates such that the sulfated precipitates has a sulfate ion content of 3.0 wt % based on the weight of the dried precipitates, and altering the amount of platinum impregnation such that the platinum content of the catalyst is 2.5 wt % based on the weight of the calcined 3.0SZA catalyst. The sample catalyst made in this example is denoted as 2.5Pt/3.0SZA.

TABLE 3
Comparisons of components and specific surface area of each catalyst

| | | | | BET |
|---|---|---|---|---|
| | | | | specific surface |
| Sample name | S (wt %) | Pt (wt %) | Al (wt %) | area (m²/g) |
| 0.3Pt/1.5SZA | 0.54 | 0.32 | 1.25 | 74.4 |
| 0.3Pt/3.0SZA | 0.99 | 0.30 | 1.21 | 80.5 |
| 0.3Pt/9.0SZA | 1.44 | 0.28 | 1.21 | 97.3 |
| 1.0Pt/3.0SZA | 1.06 | 0.90 | 1.24 | 70.5 |
| 1.5Pt/3.0SZA | 0.98 | 1.55 | 1.24 | 71.9 |
| 2.0Pt/3.0SZA | 0.99 | 2.07 | 1.23 | 73.7 |
| 2.5Pt/3.0SZA | 0.98 | 2.71 | 1.20 | 75.3 |
| 0.3Pt/1.5SZ | 0.57 | 0.31 | — | 53.4 |
| 0.3Pt/3.0SZ | 0.86 | 0.32 | — | 74.8 |
| 0.3Pt/9.0SZ | 1.06 | 0.29 | — | 84.2 |

The components and BET specific surface areas of the sample catalysts described above are measured via methods known by people with ordinary skill in the art, and the measurement data are shown in Table 3. The series of “yPt/xSZ catalysts” used herein includes 0.3Pt/1.5SZ catalyst, 0.3Pt/3.0SZ catalyst and 0.3Pt/9.0SZ catalyst; the series of “yPt/xSZA catalysts” used herein comprises 0.3Pt/1.5SZA catalyst, 0.3Pt/3.0SZA catalyst, 0.3Pt/9.0SZA catalyst, 1.0Pt/3.0SZA catalyst, 1.5Pt/3.0SZA catalyst, 2.0Pt/3.0SZA catalyst and 2.5Pt/3.0SZA catalyst

### Examples

### Analysis of Experimental Results of Isomerization Reaction

The sample catalysts as described above are used in n-paraffin (such as n-hexanes and/or n-heptanes) isomerization. The steps, parameters and results of n-paraffin isomerization are described in detail below. In this part, a commercial C₅/C₆ catalyst (marketed by SINOPEC) is used as the control.

I. Steps of the n-Paraffin Isomerization Reaction
a. providing a reaction system, wherein the reaction system has a tube and a back valve; setting the pressure of the back valve within the reaction system at 2.5 Mpa, then detecting the leak of the tube and the reaction system;
b. decreasing the pressure of the reaction system to the atmospheric pressure and separating the tube from the reaction system; removing the top connector mounted on the tube; filling the tube with 0.1 to 1.0 grams of one sample catalyst to be tested, then putting the top connector back to the tube and settling the tube back to the reaction system;
c. setting pressure of the back valve within the reaction system at 2.5 Mpa and detecting the leak of the tube and the reaction system again to assure the tube and the reaction system are both airtight;
d. decreasing the pressure of the reaction system to the atmospheric pressure; providing an air flow into the reaction system; increasing the temperature of the reaction system to about 450° C. at a rate of 10° C./min and maintaining the temperature at 450° C. for 3 hours, then decreasing to 250° C.;
e. providing a nitrogen flow into the reaction system and blowing for about 1 hour;
f. providing a hydrogen flow into the reaction system to reduce the catalyst for about 1 hour;
g. cooling down the reduced catalyst to room temperature, then adjusting the pressure of the back valve to 2.1 Mpa; providing a reaction gas (hydrogen is used herein) flow into the reaction system and analyzing the components of discharge from the reaction system by gas chromatography (GC);
h. increasing the temperature of the reaction system to a proper degree fit for the tested sample catalyst when the components of the discharge becomes stable, then testing the activity of the tested sample catalyst;
i. setting the weight hourly space velocity (WHSV) of n-hexanes or n-heptane for 2.32 h⁻¹;
j. setting the molar ratio of the feed (i.e. n-hexane and/or n-heptane)/reaction gas (i.e. H₂) to be 1:6.1.

II. Analysis and Comparison of Each Catalyst Sample Used in the n-Paraffin Isomerization Reaction while the Feed is n-Hexane and/or n-Heptane

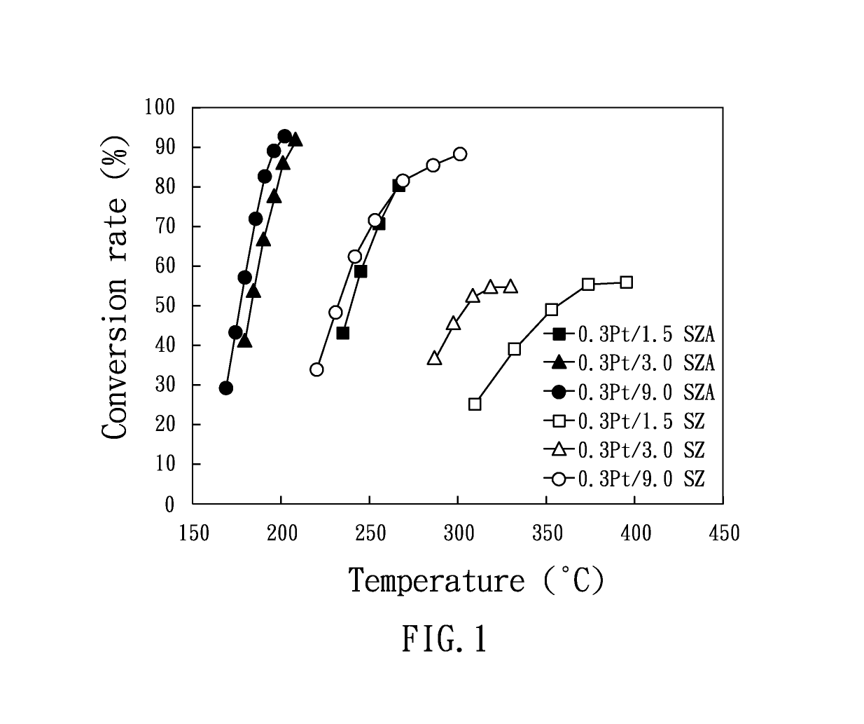

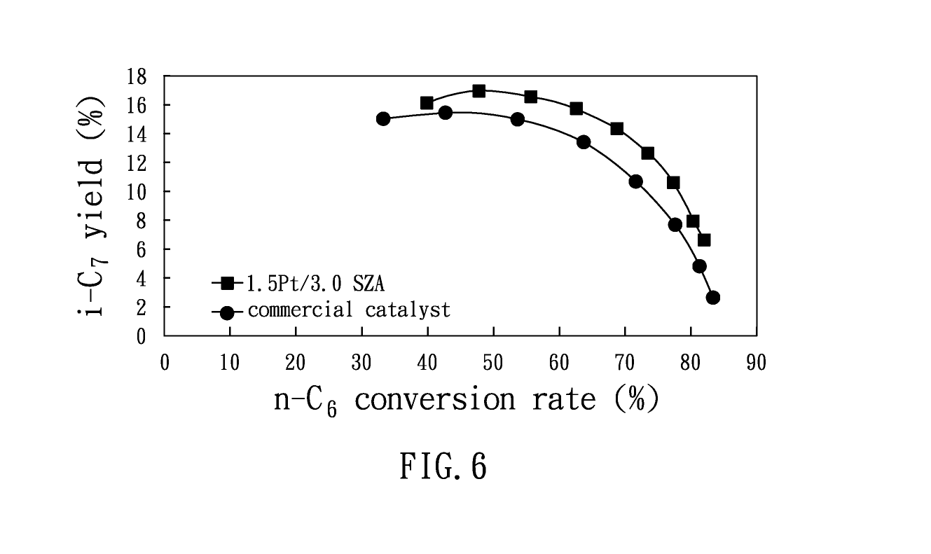

The overall conversion rate (% conversion), the i-C₇ selectivity, the n-C₆ conversion rate and isoparaffin yield of those sample catalysts during isomerization processes are calculated. FIGS. 1 to 5 show the overall conversion rate versus the reaction temperature and i-C₇ selectivity respectively. FIGS. 6 to 8 show the isoparaffin yield versus i-C₆ selectivity. The definition and calculation methods of conversion rate, selectivity and yield can be referred to the reference document below: H. Scott Fogler, Elements of Chemical Reaction Engineering, 3^{rd} Ed., Upper Saddle River, N.J.: Prentice Hall, 1999.

FIG. 1 shows the change in the overall conversion rate of the sample catalysts made in comparative examples 1 to 3 and examples 1 to 3 during n-heptane isomerization, wherein the change in the overall conversion rate is closely related with the change of the activity of the catalysts. Higher conversion rate means higher activity under the same temperature.

As shown in FIG. 1, a sample catalyst having less content of sulfate impregnation possesses lower activity and requires higher reaction temperature. The overall conversion rate of 0.3Pt/1.5SZ catalyst and 0.3Pt/3.0SZ catalyst are only up to about 55% as used in n-heptane isomerization, even though the temperature is raised to 390° C. When the reaction temperature in the system is as high as 300° C., the sulfate of the two sample catalysts may react with H₂ to form H₂S such that the sulfate content of the two sample catalysts is decreasing and thus resulting in degressive activity over time. Therefore, increasing reaction temperature cannot improve the overall conversion rate of such catalysts effectively but causes loss of the sulfate content as well as loss of activity of the catalysts instead.

In the case of same amount of sulfate impregnation, the activity of the yPt/xSZA catalysts is obviously greater than the yPt/xSZ catalysts. At the same overall conversion rate, the yPt/xSZA catalysts need a reaction temperature about 60° C. lower than that of the yPt/xSZ catalysts. The activity of the yPt/xSZA catalysts is rising with the increase of the sulfate content, for example, the activity of the 0.3Pt/3.0SZA catalyst is higher than that of 0.3Pt/1.5SZA catalyst, and the reaction temperature of 0.3Pt/3.0SZA catalyst is about 55° C. lower than that of 0.3Pt/1.5SZA catalyst under the same conversion rate.

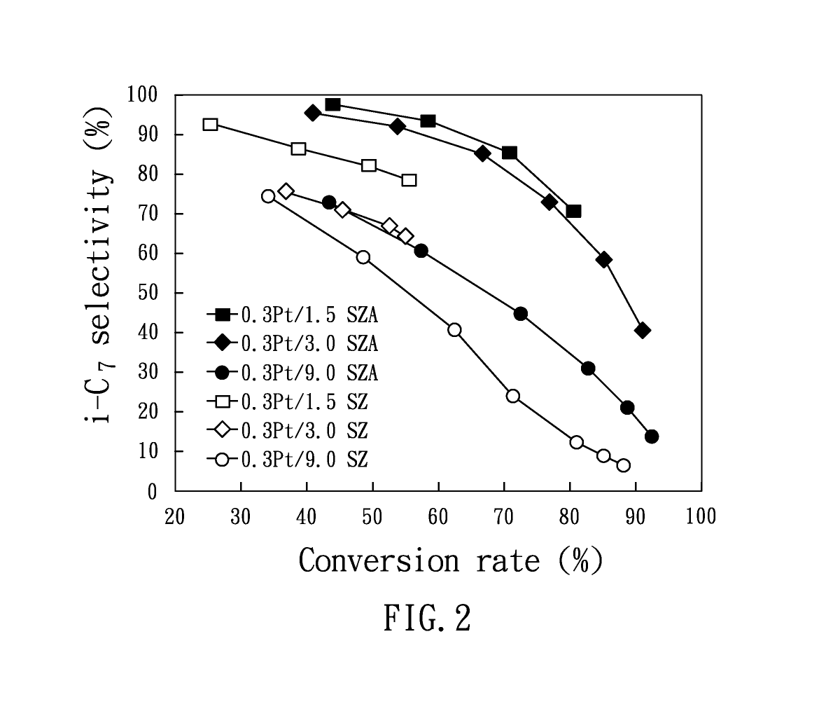

FIG. 2 shows the i-C₇ selectivity of the sample catalysts made in comparative examples 1 to 3 and examples 1 to 3 respectively versus the overall conversion rate thereof during n-heptane isomerization. As shown in FIG. 2, the i-C₇ selectivity of the yPt/xSZ catalysts is increasing with the decrease of the sulfate content, wherein the 0.3Pt/1.5SZ catalyst has considerably high i-C₇ selectivity, but its conversion rate is only up to 55%. The i-C₇ selectivity of the yPt/xSZA catalysts is mostly higher than that of the yPt/xSZ catalysts, among which the 0.3Pt/1.5SZA catalyst and the 0.3Pt/3.0SZA catalyst are particularly the highest two in i-C₇ selectivity, and the i-C₇ selectivity of the 0.3 Pt/3.0SZA catalyst is a little lower than that of the 0.3Pt/1.5SZA catalyst; however, the activity of the 0.3Pt/3.0SZA catalyst is much higher than that of the 0.3Pt/1.5SZA catalyst. The i-C₇ selectivity of the 0.3Pt/1.5SZA catalyst and the 0.3 Pt/3.0SZA catalyst are both higher than 83% under a conversion rate of 70% and the i-C₇ selectivity of the 0.3Pt/9.0SZA catalyst is lower than that of the 0.3Pt/1.5SZA catalyst and the 0.3 Pt/3.0SZA catalyst (only 50%). The i-C₇ selectivity of the 0.3Pt/9.0SZ catalyst is only 25%.

FIG. 2 shows that the sample catalysts with low amount of sulfate impregnation (such as 0.3Pt/1.5SZA and 0.3Pt/3.0SZA) mostly have good i-C₇ selectivity. Thus, it is understood that adjusting the amount of sulfate impregnation to less than or equal to 3.0 wt % (based on the weight of the dried precipitates) during catalysts preparation can make the produced catalysts have good activity and good selectivity of isoparaffin, especially when the amount of sulfate impregnation is equal to 3.0 wt %.

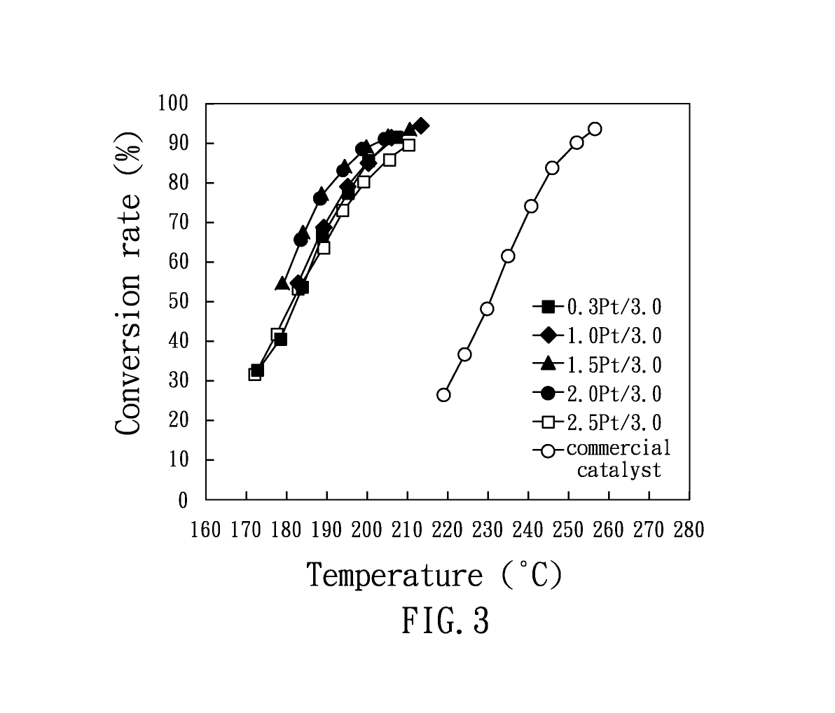

FIG. 3 shows the overall conversion rate of the catalysts made in examples 4 to 7 and of the commercial catalyst respectively versus the reaction temperature during n-heptane isomerization, wherein the yPt/xSZA catalysts made in examples 4 to 7 all have 3.0 wt % of the amount of the sulfate impregnation (based on the weight of the dried precipitates) for contrasting the activity of the yPt/xSZA catalysts respectively with different platinum contents and the commercial catalyst.

As shown in FIG. 3, the 0.3Pt/3.0SZA catalyst, 1.0Pt/3.0SZA catalyst, 1.5Pt/3.0SZA catalyst, 2.0Pt/3.0SZA catalyst and 2.5Pt/3.0SZA catalyst all have higher activity than the commercial catalyst, especially the 1.5Pt/3.0SZA catalyst and 2.0Pt/3.0SZA catalyst. In the case of the same overall conversion rate, the reaction temperature of the yPt/xSZA catalysts is 50° C. lower than that of the commercial catalyst.

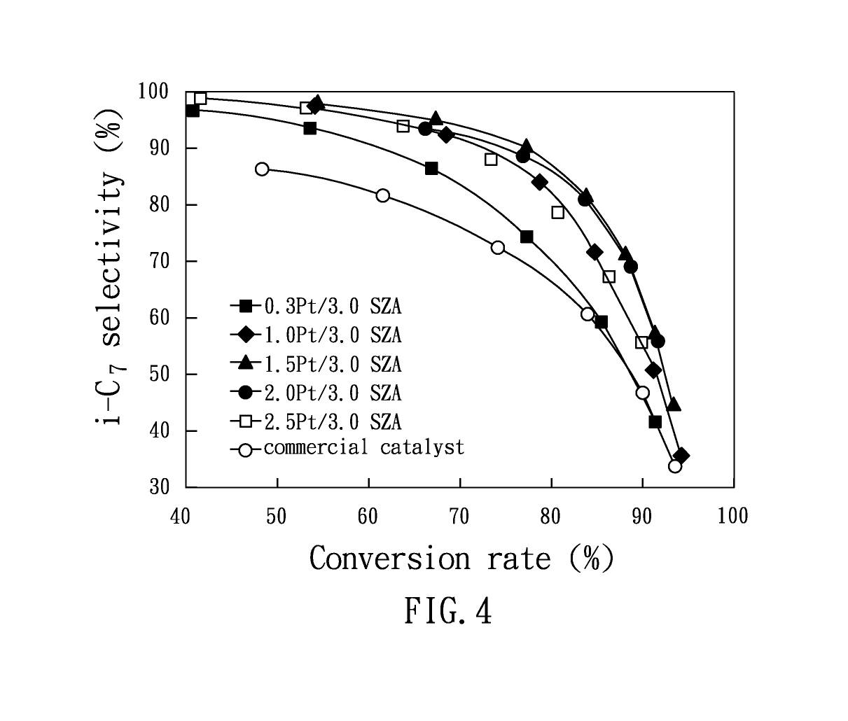

FIG. 4 shows the i-C₇ selectivity at different overall conversion rates of the catalysts made in examples 4 to 7 and the commercial catalyst during n-heptane isomerization, wherein the i-C₇ selectivity of 0.3Pt/3.0SZA catalyst, 1.0Pt/3.0SZA catalyst, 1.5Pt/3.0SZA catalyst, 2.0Pt/3.0SZA and 2.5Pt/3.0SZA catalyst is better than that of the commercial catalyst, especially of 1.5Pt/3.0SZA catalyst and 2.0Pt/3.0SZA catalyst. At a conversion rate of 80%, the i-C₇ selectivity of the yPt/xSZA catalysts is up to 87%; on the contrary, the i-C₇ selectivity of the commercial catalyst is only up to 67%.

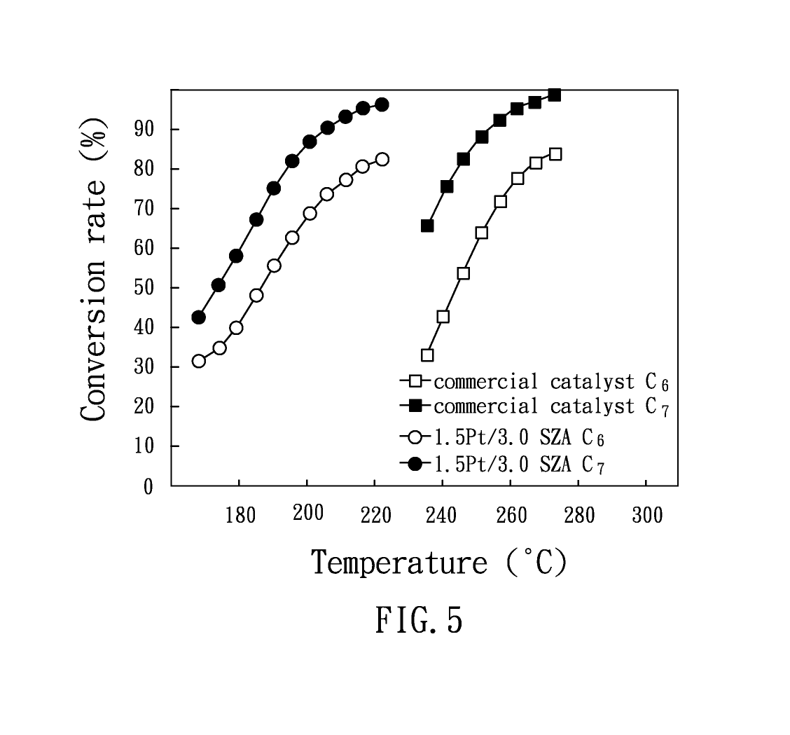

FIG. 5 shows the activity of the commercial catalyst and the 1.5Pt/3.0SZA catalyst as the feed contains n-hexane and n-heptane (the volume ratio of C₆/C₇: 70/30) during isomerization process. As shown in FIG. 5, the two catalysts both have higher activity in n-hexane isomerization than n-heptane isomerization; however, the reaction temperature of the 1.5Pt/3.0SZA catalyst is 50° C. lower than that of the commercial catalyst under the same conversion rate.

FIGS. 6 to 8 show the amount of i-C₇ product obtained as each sample catalyst is applied to isomerization processes and the feed used herein is for the most part n-hexanes with a small quantity of n-heptanes in order to simulate the actual feed.

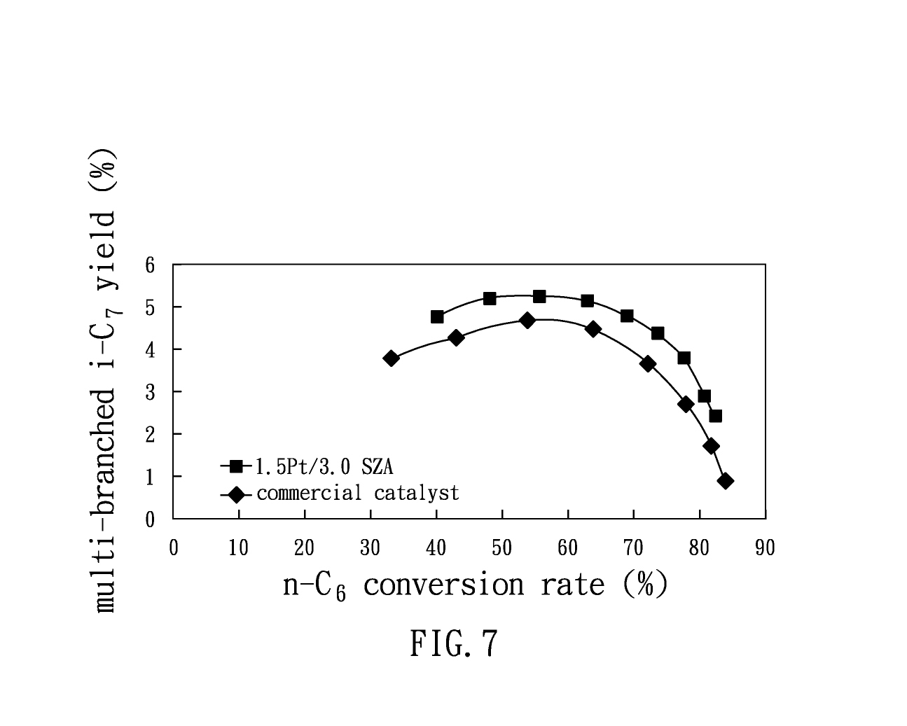

FIG. 6 shows the i-C₇ product yields of the isomerization with 1.5Pt/3.0SZA catalyst and the commercial catalyst respectively at different conversion rates of n-hexane isomerization. As shown in FIG. 7, the i-C₇ product yield of the isomerization with 1.5Pt/3.0SZA catalyst is up to 17% and higher than that of the commercial catalyst.

FIG. 7 shows the multi-branched i-C₇ product yields while using the 1.5Pt/3.0SZA catalyst and the commercial catalyst in n-hexane isomerization respectively versus the n-C₆ conversion rate. As shown in FIG. 7, the n-hexane isomerization using the 1.5Pt/3.0SZA catalyst can obtain a higher multi-branched i-C₇ product yield.

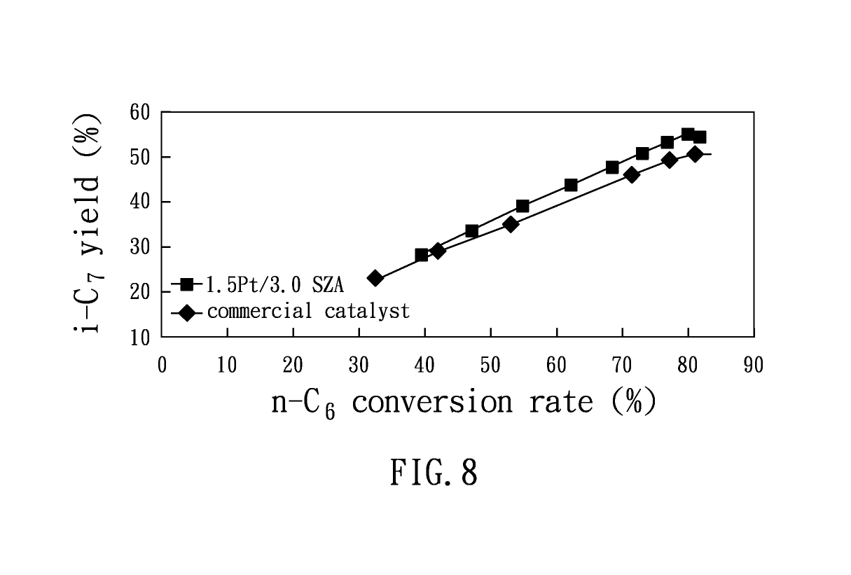

FIG. 8 shows the i-C₇ product yields of the isomerization with 1.5Pt/3.0SZA catalyst and the commercial catalyst. The two catalysts have substantially identical i-C₇ product yield under low n-C₆ conversion rate; however, use of the 1.5Pt/3.0SZA catalyst leads to higher i-C₇ product yield in the isomerization than that of the commercial catalyst at high n-C₆ conversion rate. Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A process for converting paraffin comprising the steps of:
(i) providing feed containing n-pentane, n-hexane and more than 2 vol % n-heptane based on the volume of the feed; and
(ii) subjecting the feed to isomerization of n-heptane with a modified zirconia catalyst, wherein the modified zirconia catalyst comprises zirconium oxide, sulfate ions, a first metal component and a second metal component, the first metal component contains at least one of Group III A (IUPAC 13) metal elements or a combination thereof at an amount of between 0.1 wt % and 15 wt % based on the weight of the catalyst, the second metal component contains a substance selected from a group consisting of platinum, platinum oxide, palladium, palladium oxide and a combination thereof at an amount of between 0.2 wt % and 3.0 wt % based on the weight of the catalyst, and sulfate ions contain sulfur at an amount of less than 1.0 wt % based on the weight of the catalyst;
wherein the i-C₇ selectivity is higher than 80% as the conversion rate rises to 80%.

2. The process for converting paraffin as claimed in claim 1, wherein a source of the sulfate anions comprises ammonium sulfate, sulfuric acid or a combination thereof.

3. The process for converting paraffin as claimed in claim 1, wherein the amount of the first metal component is between 0.1 wt % and 10 wt % based on the weight of the catalyst.

4. The process for converting paraffin as claimed in claim 3, wherein the first metal component comprises a substance selected from the group consisting of aluminum, gallium and a combination thereof.

5. The process for converting paraffin as claimed in claim 1, wherein the second metal component is platinum.

6. The process for converting paraffin as claimed in claim 1, wherein the zirconium oxide is ZrO₂.

7. The process for converting paraffin as claimed in claim 1, wherein the BET specific surface area of the catalyst ranges from 50 m²/g to 130 m²/g.

8. The process for converting paraffin as claimed in claim 2, wherein the BET specific surface area of the catalyst ranges from 50 m²/g to 130 m²/g.

9. The process for converting paraffin as claimed in claim 3, wherein the BET specific surface area of the catalyst ranges from 50 m²/g to 130 m²/g.

10. The process for converting paraffin as claimed in claim 4, wherein the BET specific surface area of the catalyst ranges from 50 m²/g to 130 m²/g.

11. The process for converting paraffin as claimed in claim 5, wherein the BET specific surface area of the catalyst ranges from 50 m²/g to 130 m²/g.

12. The process for converting paraffin as claimed in claim 6, wherein the BET specific surface area of the catalyst ranges from 50 m²/g to 130 m²/g.
